(19) United States
(12) Patent Application Publication
(10) Pub. No.: US 2014/0170744 A1
(43) Pub. Date: Jun. 19, 2014

(54) CELL CULTURE DEVICE

(71) Applicant: SAMSUNG ELECTRO-MECHANICS CO., LTD., Suwon (KR)

(72) Inventors: Sang Jin KIM, Gyunggi-do (KR); Bo Sung KU, Gyunggi-do (KR)

(73) Assignee: SAMSUNG ELECTRO-MECHANICS CO., LTD., Suwon (KR)

(21) Appl. No.: 13/784,024

(22) Filed: Mar. 4, 2013

(30) Foreign Application Priority Data
Dec. 17, 2012 (KR) 10-2012-0147565

(51) Int. Cl. C12Q 1/02

(52) U.S. Cl. CPC C12Q 1/025
USPC 435/297.2; 435/289.1

(57) ABSTRACT

There is provided a cell culture device including a cell chip accommodation unit accommodating a cell chip therein, a drug storage unit storing a drug and having a closed structure, a first pipe connecting the drug storage unit and the cell chip accommodation unit and transferring the drug from the drug storage unit to the cell chip accommodation unit, a second pipe connecting the drug storage unit and the cell chip accommodation unit and transferring the drug from the cell chip accommodation unit to the drug storage unit, and a pump circulating the drug between the cell chip accommodation unit and the drug storage unit.

### CROSS-REFERENCE TO RELATED APPLICATIONS

This application claims the priority of Korean Patent Application No. 10-2012-0147565 filed on Dec. 17, 2012, in the Korean Intellectual Property Office, the disclosure of which is incorporated herein by reference.

### BACKGROUND OF THE INVENTION

1. Field of the Invention

The present invention relates to a cell culture device, and more particularly, to a cell culture device capable of smoothly circulating a drug.

2. Description of the Related Art

Demand for bio medical devices and bio technologies for promptly diagnosing various human diseases has recently increased. Accordingly, the development of experimental devices and instruments capable of providing diagnosis results in a relatively short time, as compared with existing, relatively time-consuming methods of diagnosing diseases in hospitals and laboratories has been actively conducted.

In order to develop new medicines and perform experiments to determine the stability of new medicines, it is essential to observe a reaction between a new medicine (i.e. a drug) and cells. In general, an experiment for determining a reaction between a drug and cells is performed by using a culture plate, or the like.

However, since a reaction between a drug and cells conducted in a culture plate may be quite different from a reaction between drug and cells occurring inside a body, it is difficult to exactly observe or inspect the reaction between a drug and cells based only on a result of an experiment using the culture plate. Thus, development of a culturing device for circulating a drug so as to observe the reaction between drug and cells in an environment similar to that of an interior of a body is required.

The prior art regarding circulation of drug includes Patent Documents 1 and 2. These patent documents disclose a technology of circulating a drug in a culture medium tank 20 and culturing a sample such as a plant cell.

However, since Patent Documents 1 and 2 disclose a device for circulating a large quantity of a drug, it is difficult to precisely and regularly circulate drug in units of several μl˜several tens of μl.

### RELATED ART DOCUMENTS

(Patent Document 1) JP No. 1993-336945 A
(Patent Document 2) JP No. 1993-276922 A

### SUMMARY OF THE INVENTION

An aspect of the present invention provides a cell culture device capable of precisely and regularly circulating a small quantity of a drug.

Another aspect of the present invention provides a cell culture device capable of precisely and regularly circulating a small quantity of a drug, promoting smooth culturing of a cell, and increasing reliability of an influence evaluation of drugs.

According to an aspect of the present invention, there is provided a cell culture device including: a cell chip accommodation unit accommodating a cell chip therein; a drug storage unit storing a drug and having a closed structure; a first pipe connecting the drug storage unit and the cell chip accommodation unit and transferring the drug from the drug storage unit to the cell chip accommodation unit; a second pipe connecting the drug storage unit and the cell chip accommodation unit and transferring the drug from the cell chip accommodation unit to the drug storage unit; and a pump circulating the drug between the cell chip accommodation unit and the drug storage unit.

The first pipe may be connected to a lower portion of the drug storage unit, and the second pipe may be connected to an upper portion of the drug storage unit.

The first pipe maybe connected to one side of the cell chip accommodation unit, and the second pipe may be connected to the other side of the cell chip accommodation unit.

The first pipe may be connected to a first connection tube formed in a bottom of the cell chip accommodation unit, and the second pipe maybe connected to a second connection tube formed in the bottom of the cell chip accommodation unit.

The first connection tube and the second connection tube may have an average diameter greater than an average diameter of the first pipe and the second pipe.

The cell chip accommodation unit may include a flow path determining a transfer path of the drug.

The flow path may have a zigzag shape.

The drug storage unit may be divided into a plurality of drug storage spaces such that the same or different kinds of drug are stored separately.

The cell chip accommodation unit may be divided into a plurality of regions.

The plurality of regions and the plurality of drug storage spaces may be equal in number.

At least one of the cell chip accommodation unit and the drug storage unit may include a filter filtering foreign materials contained in the drug circulating between the cell chip accommodation unit and the drug storage unit.

The drug storage unit may include a cover or a film member covering one surface thereof.

According to another aspect of the present invention, there is provided a cell culture device including: a cell chip accommodation unit accommodating a cell chip therein; a drug storage unit storing a drug and having a hydraulic head lower than that of the cell chip accommodation unit; a first pipe connecting the drug storage unit and the cell chip accommodation unit and transferring the drug from the drug storage unit to the cell chip accommodation unit; a second pipe connecting the drug storage unit and the cell chip accommodation unit and transferring the drug from the cell chip accommodation unit to the drug storage unit; and a pump circulating the drug between the cell chip accommodation unit and the drug storage unit.

The first pipe may be connected to a lower portion of the drug storage unit, and the second pipe may be connected to an upper portion of the drug storage unit.

The first pipe maybe connected to one side of the cell chip accommodation unit, and the second pipe may be connected to the other side of the cell chip accommodation unit.

The first pipe may be connected to a first connection tube formed in a bottom of the cell chip accommodation unit, and the second pipe maybe connected to a second connection tube formed in the bottom of the cell chip accommodation unit.

The first connection tube and the second connection tube may have an average diameter greater than an average diameter of the first pipe and the second pipe.

The cell chip accommodation unit may include a flow path determining a transfer path of the drug.

The flow path may have a zigzag shape.

The drug storage unit may be divided into a plurality of drug storage spaces such that the same or different kinds of drug may be stored separately.

The cell chip accommodation unit may be divided into a plurality of regions.

The plurality of regions and the plurality of drug storage spaces may be equal in number.

At least one of the cell chip accommodation unit and the drug storage unit may include a filter filtering foreign materials contained in the drug circulating between the cell chip accommodation unit and the drug storage unit.

The drug storage unit may include a cover or a film member covering one surface thereof.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a view of a cell culture device according to an embodiment of the present invention;

FIGS. 2 through 4 are plan views showing modifications of a cell chip accommodation unit of FIG. 1;

FIG. 5 is a cross-sectional view of a cell culture unit taken along line A-A of FIG. 1;

FIG. 6 is a cross-sectional view of a drug storage unit taken along line B-B of FIG. 1;

FIG. 7 is a view of the cell culture device having a cell chip mounted therein according to the embodiment of the present invention;

FIG. 8 is a cross-sectional view taken along line B-B of FIG. 7;

FIG. 9 is a cross-sectional view of the cell culture device having a drug circulation structure according to the embodiment of the present invention;

FIG. 10 is a cross-sectional view of a cell culture device according to another embodiment of the present invention; and

FIGS. 11 and 12 are views respectively showing a cell culture device according to another embodiment of the present invention.

### DETAILED DESCRIPTION OF THE EMBODIMENTS

Hereinafter, embodiments of the present invention will be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art.

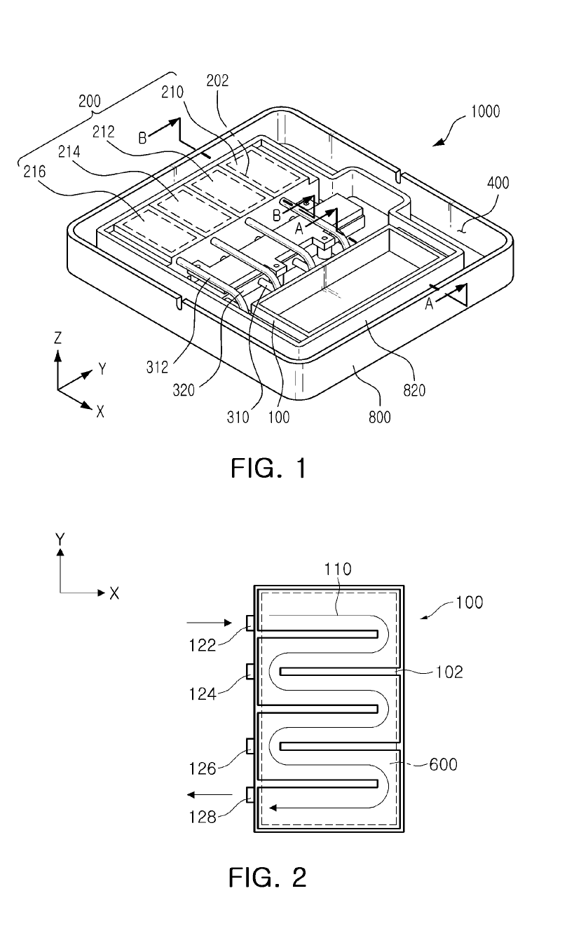

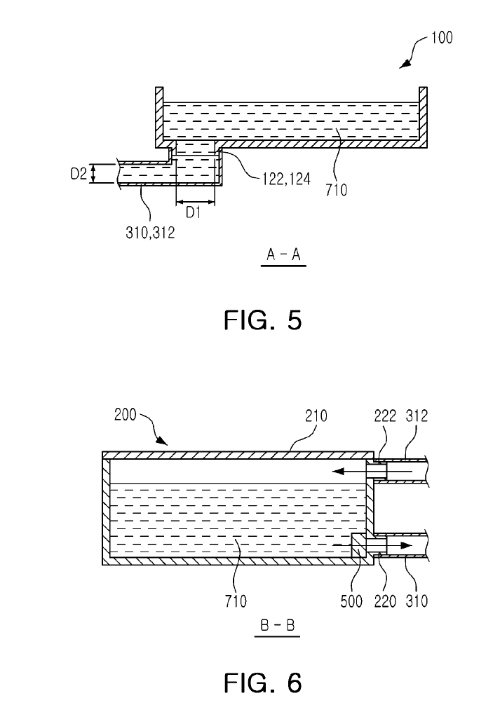

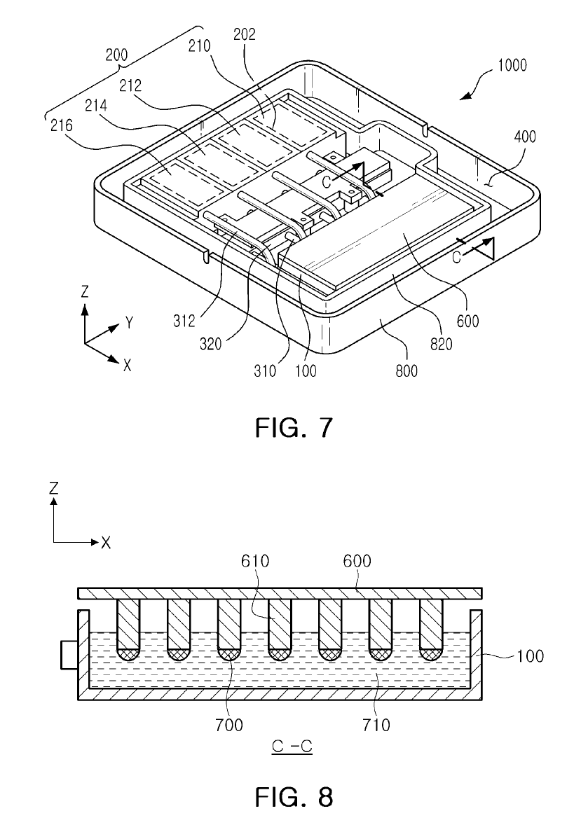

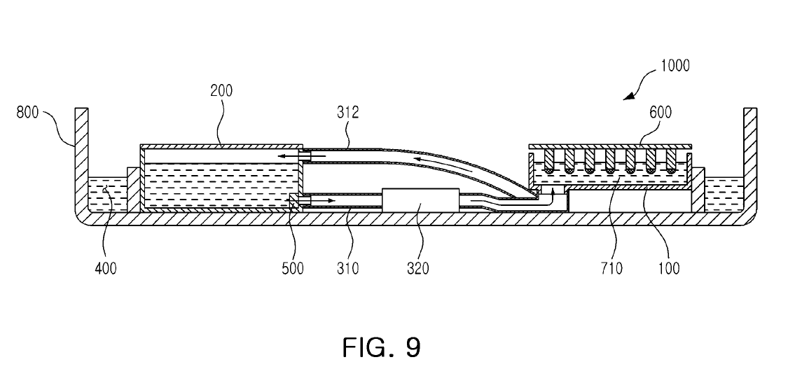

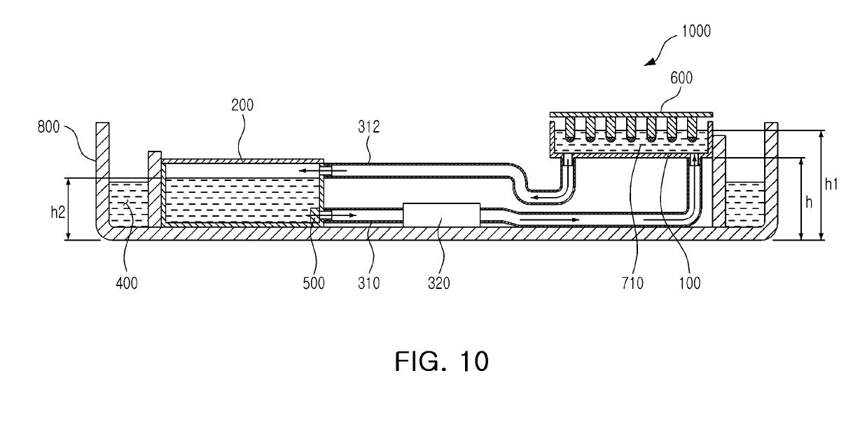

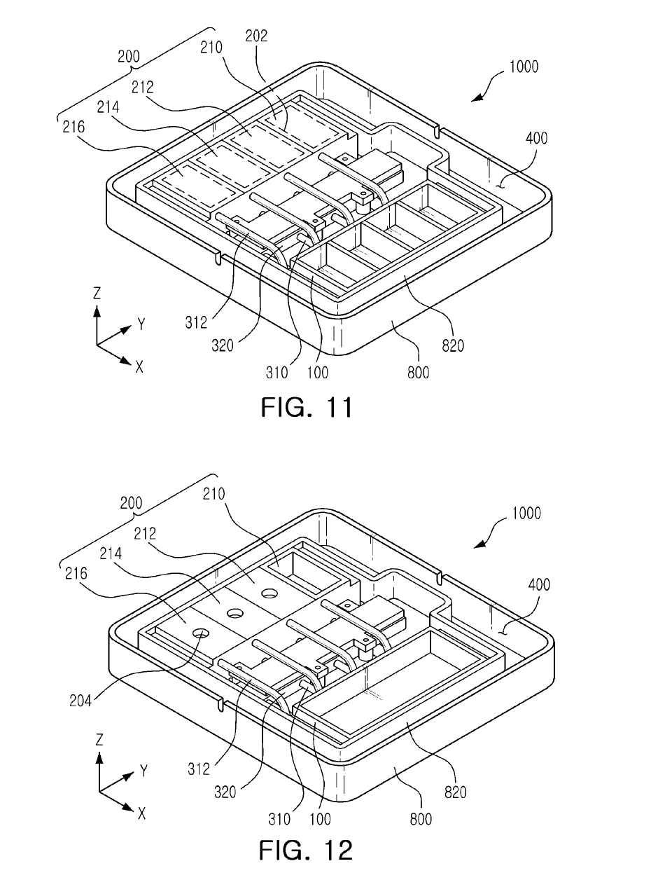

FIG. 1 is a view of a cell culture device according to an embodiment of the present invention. FIGS. 2 through 4 are plan views showing modifications of a cell chip accommodation unit of FIG. 1. FIG. 5 is a cross-sectional view of a cell culture unit taken along line A-A of FIG. 1. FIG. 6 is a cross-sectional view of a drug storage unit taken along line B-B of FIG. 1. FIG. 7 is a view of the cell culture device having a cell chip mounted therein according to the embodiment of the present invention. FIG. 8 is a cross-sectional view taken along line B-B of FIG. 7. FIG. 9 is a cross-sectional view of the cell culture device having a drug circulation structure according to the embodiment of the present invention. FIG. 10 is a cross-sectional view of a cell culture device according to another embodiment of the present invention. FIGS. 11 and 12 are views respectively showing a cell culture device according to another embodiment of the present invention.

A cell culture device 1000 according to an embodiment of the present invention will now be described with reference to FIGS. 1 through 9.

The cell culture device 1000 may include a cell chip accommodation unit 100, a drug storage unit 200, first and second pipes 310 and 312, and a pump 320. In this regard, the cell chip accommodation unit 100 and the drug storage unit 200 may be integrally formed in a single body 800. However, the cell chip accommodation unit 100 and the drug storage unit 200 are not necessarily integrally formed with the body 800. For example, at least one of the cell chip accommodation unit 100 and the drug storage unit 200 may be separated from the body 800.

The cell chip accommodation unit 100 may accommodate at least one cell chip therein. To this end, the cell chip accommodation unit 100 may include a space for accommodating the cell chip. The cell chip may be mounted in the space in a state in which it is overturned (see FIG. 8).

The cell chip accommodation unit 100 may include a plurality of first connection tubes 122, 124, 126, and 128 for connecting the cell chip accommodation unit 100 to a circulation device 300 (see FIG. 2). The first connection tubes 122, 124, 126, and 128 may be formed in one direction (in a Y-axis direction of FIG. 1) of the cell chip accommodation unit 100. In this regard, positions of the first connection tubes 122, 124, 126, and 128 and intervals therebetween are not particularly limited. For example, the intervals between the first connection tubes 122, 124, 126, and 128 may be the same as one another, or may be partially different. In addition, the number of the first connection tubes 122, 124, 126, and 128 is not particularly limited. For example, FIG. 2 shows that the four first connection tubes 122, 124, 126, and 128 are formed in the cell chip accommodation unit 100; however, the number of the first connection tubes may be increased or decreased as necessary.

Meanwhile, the cell chip accommodation unit 100 may have a flow path 110 inducing the flow (movement) of a drug. More specifically, the flow path 110 may be formed by a partition 102 partially dividing the cell chip accommodation unit 100. Through the flow path 110, a drug introduced into the cell chip accommodation unit 100 may be induced to be sequentially in contact, or react with, at least one kind of bio materials attached to the cell chip.

Various modifications of the flow path 110 will now be described with reference to FIGS. 2 through 4.

A modification of the flow path 110 may be formed to have a zigzag shape by the partition 102 extending in an X-axis direction as shown in FIG. 2. In this case, the first connection tubes 122 and 128 of the cell chip accommodation unit 100 may be connected to the first and second pipes 310 and 312, respectively, and the remaining first connection tubes 124 and 126 thereof may be closed. The flow path 110 having the above-described form may be appropriate for a case in which different kinds of bio materials are arranged in a Y-axis direction.

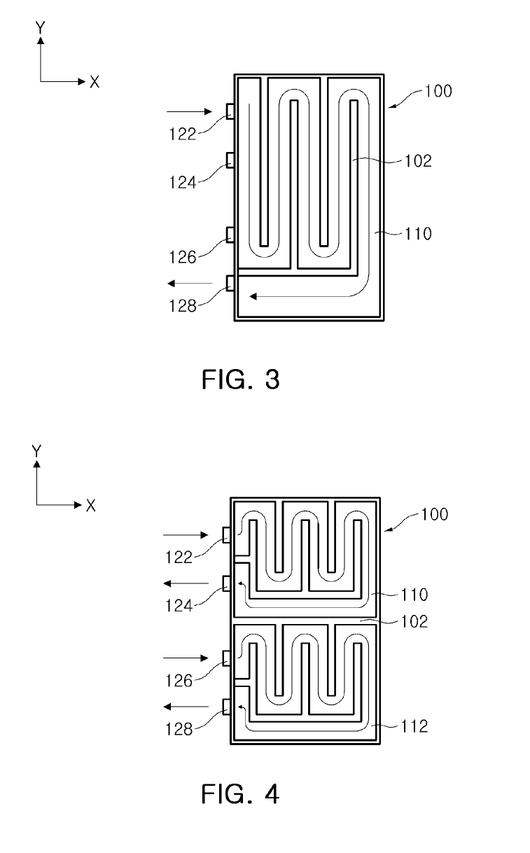

Another modification of the flow path 110 maybe formed to have a zigzag shape by the partition 102 extending in the Y-axis direction as shown in FIG. 3. In this case, one of the first connection tubes 122, 124, and 126 of the cell chip accommodation unit 100 may be connected to the first pipe 310, and the first connection tube 128 thereof may be connected to the second pipe 312. The flow path 110 having the above-described form may be appropriate for a case in which different kinds of bio materials are arranged in the X-axis direction.

According to another modification of flow paths 110 and 112, the flow path may be divided into two regions as shown in FIG. 4. More specifically, one portion of the cell chip accommodation unit 100 may be provided with a first flow path 110 connected from the first connection tube 122 to the first connection tube 124, and the other portion thereof may be provided with a second flow path 112 connected from the first connection tube 126 to the first connection tube 128. The flow paths 110 and 112 as described above may be appropriate for an experiment on different kinds of drug with regard to a single cell chip.

Meanwhile, the first and second connection tubes 122 and 124 may be formed in the bottom of the cell chip accommodation unit 100 as shown in FIG. 5. In this case, a diameter D1 of the first and second connection tubes 122 and 124 maybe greater than the diameter thereof in the case in which the first and second connection tubes 122 and 124 are formed in a side surface of the cell chip accommodation unit 100, and thus, a drug may readily flow through the first and second connection tubes 122 and 124. In addition, if the first and second connection tubes 122 and 124 are formed in the bottom of the cell chip accommodation unit 100, it maybe advantageous to uniformly maintain a level of the drug introduced into the cell chip accommodation unit 100.

The diameter D1 of the first and second connection tubes 122 and 124 may be greater than a diameter D2 of the first and second pipes 310 and 312. In this case, flow resistance may be reduced in connection portions of the first and second connection tubes 122 and 124 and the first and second pipes 310 and 312.

For reference, the first and second connection tubes 122 and 124 are merely illustrated in FIG. 5; however, the third and fourth connection tubes 126 and 128 may be formed in the bottom of the cell chip accommodation unit 100 in a similar manner to the first and second connection tubes 122 and 124.

The drug storage unit 200 may store the drug. In more detail, the drug storage unit 200 may include at least one or more drug storage spaces 210, 212, 214, and 216. Each of the drug storage spaces 210, 212, 214, and 216 may be divided by the partition 202. In this regard, the same kind of drug or different kinds thereof may be stored in the respective drug storage spaces 210, 212, 214, and 216. As an example, the same kind of drug may be stored in the respective first drug storage space 210, the second drug storage space 212, the third drug storage space 214, and the fourth drug storage space 216. As another example, different kinds of drug may be stored in the respective first drug storage space 210, the second drug storage space 212, the third drug storage space 214, and the fourth drug storage space 216. As another example, the same kind of drug may be stored in the first drug storage space 210, the second drug storage space 212, and different kinds of drug maybe stored in the third drug storage space 214, and the fourth drug storage space 216. However, the forms in which the drugs are stored are not limited to the above-mentioned three examples, but may be varied as necessary. Meanwhile, FIG. 1 shows that the drug storage unit 200 is divided into four drug storage spaces 210, 212, 214, and 216; however, the number of drug storage spaces 210, 212, 214, and 216 may be increased or decreased as necessary.

The drug storage unit 200 may have a volume equal to or larger than that of the cell chip accommodation unit 100. For example, the drug storage unit 200 may have sufficient volume so that a predetermined amount of drugs are circulated between the cell chip accommodation unit 100 and the drug storage unit 200. Meanwhile, FIG. 1 shows that each volume of the drug storage spaces 210, 212, 214, and 216 is smaller than that of the cell chip accommodation unit 100; however, each volume of the drug storage spaces 210, 212, 214, and 216 may be equal to or larger than that of the cell chip accommodation unit 100 as necessary.

The drug storage unit 200 may include a plurality of second connection tubes 220 and 222 connected to the pipes 310 and 312. The second connection tubes 220 and 222 may be formed in each of the drug storage spaces 210, 212, 214, and 216. In this regard, the second connection tube 220 may be used as an outlet from which the drug is discharged, and the second connection tube 222 may be used as an inlet into which the discharged drug is reintroduced.

The drug storage unit 200 may be connected to the cell chip accommodation unit 100 by the first and second pipes 310 and 312. That is, the drug of the drug storage unit 200 may be supplied to the cell chip accommodation unit 100 by the first pipe 310, and the drug of the cell chip accommodation unit 100 maybe reintroduced into the drug storage unit 200 by the second pipe 312. In this regard, pressure required for circulating the drug may be provided by the pump 320.

The above-configured drug storage unit 200 may be disposed to face the cell chip accommodation unit 100. For example, the drug storage unit 200 may be disposed to be symmetrical with regard to the cell chip accommodation unit 100 based on the Y-axis as shown in FIG. 1. However, the arrangement of the drug storage unit 200 is not limited to the embodiment thereof as shown in FIG. 1 and may be varied if necessary.

The drug storage unit 200 may have a closed structure. In more detail, the drug storage unit 200 may have the closed structure in which all portions are enclosed except for the second connection tubes 220 and 222 as shown in FIG. 6. In this regard, the inside of the drug storage unit 200 may be maintained at a certain pressure level. For example, the inside of the drug storage unit 200 may be maintained at a pressure lower than atmospheric pressure. In this case, the drug may be reintroduced into the drug storage unit 200 through the second pipe 312 in an amount equal to that which has been discharged from the drug storage unit 200 through the first pipe 310. Thus, the cell culture device 1000 of the present invention may circulate a drug by using one pump 320, thereby achieving simplification and miniaturization of the cell culture device 1000. Meanwhile, for an efficient circulation of drug, the first pipe 310 may be connected to a lower portion of the drug storage unit 200, and the second pipe 312 may be connected to an upper portion of the drug storage unit 200.

The first and second pipes 310 and 312 may connect the drug storage unit 200 and the cell chip accommodation unit 100. More specifically, the first connection tube 122 of the cell chip accommodation unit 100 and the second connection tube 220 of the drug storage unit 200 may be connected to one another by the first pipe 310 and the first connection tube 124 of the cell chip accommodation unit 100 and the second connection tube 222 of the drug storage unit 200 maybe connected to one another by the second pipe 312. Meanwhile, these connections are merely exemplary, and may be varied as necessary. For example, a plurality of pipes 310 and 312 may connect the one cell chip accommodation unit 100 and all of the plurality of drug storage spaces 210, 212, 214, and 216.

The pump 320 may be mounted on the first pipe 310, and provide pressure required for circulating a drug. For example, the pump 320 may be mounted on the first pipe 310 to transfer the drug from the drug storage unit 200 to the cell chip accommodation unit 100. Meanwhile, as described above, since the inside of the drug storage unit 200 is uniformly maintained at a certain pressure level, if the drug from the drug storage unit 200 is discharged through the pump 320, the inside of the drug storage unit 200 may be in a negative pressure state, and thus the drug of the cell chip accommodation unit 100 may be naturally introduced through the second pipe 312.

The first and second pipes 310 and 312 and the pump 320 maybe freely coupled to and separated from the body 800. Thus, at least one of the drug storage spaces 210, 212, 214, and 216 may be selected to conduct a drug experiment according to an experiment purpose with regard to the cell chip mounted in the cell chip accommodation unit 100 by a user.

The body 800 may include a humidity maintaining space 400. In more detail, the humidity maintaining space 400 for storing water therein may be formed between a side surface of the body 800 and a main wall 820. The water stored in the humidity maintaining space 400 may uniformly maintain surrounding humidity of the cell chip accommodation unit 100 to allow for a smooth reaction between a bio material 700 and a drug 710 and reduce a phenomenon in which the bio material 700 withers.

The cell culture device 1000 as configured above may accommodate at least one cell chip 600 in the cell chip accommodation unit 100 as shown in FIG. 7. In this regard, the cell chips 600 may be disposed in a state in which it is overturned as shown in FIG. 8. Thus, the bio material 700 adhered to a pillar 610 of the cell chip 600 may be reacted with the drug 710 supplied into the cell chip accommodation unit 100. In this regard, the drug 710 is continuously circulated between the cell chip accommodation unit 100 and the drug storage unit 200 through the pipes 310 and 312. As mentioned above, in the cell culture device 1000 in which drug circulation is performed, an environment similar to that inside a body may be provided, such that an experiment and an observation on the bio material 700 with regard to the drug 710 may be conducted for a long period of time.

In addition, the cell culture device 1000 according to the present invention may allows for various modifications of the flow path 110 in the cell chip accommodation unit 100,thereby creating an environment such as in vivo experiment or culture in vivo. Thus, an experiment and an observation with respect to an effect of the drug 710 on human beings may be relatively accurately conducted in the present cell culture device 1000.

Also, an experiment and an observation of continuous supply of the drug, waste materials accumulation, and the reaction of the bio material 700 to a change in concentration of the drug accordingly may be conducted in the cell culture device 1000.

Meanwhile, the cell culture device 1000 may include a filter 500 (see FIG. 9) mounted in the cell chip accommodation unit 100 or the drug storage unit 200, whereby foreign materials generated during the reaction between the drug 710 and the bio material 700 may be selectively removed. In addition, the foreign materials filtered by the filter 500 are collected, and the foreign materials or waste materials generated during the reaction between the drug 710 and the bio material 700 may be separately observed.

In addition, the cell culture device 1000 may smoothly circulate a drug through the action of one pump 320, thereby reducing manufacturing costs of the cell culture device 1000 as well as miniaturizing the cell culture device 1000.

Also, the cell culture device 1000 may facilitate the quantitative supply of a drug, since the drug is transferred from the drug storage unit 200 to the cell chip accommodation unit 100 according to pressure generated by the pump 320 and negative pressure of the drug storage unit 200 caused by the pressure.

Another embodiment of the present invention will now be described with reference to FIG. 10.

The present embodiment may be distinguished from the foregoing embodiment in terms of the arrangement structure of the cell chip accommodation unit 100.

In the present embodiment, a hydraulic head difference between the cell chip accommodation unit 100 and the drug storage unit 200 is additionally used in order to smoothly circulate a drug. In more detail, the cell chip accommodation unit 100 may be disposed to be higher than the drug storage unit 200. For example, a height h from a bottom surface of the body 800 to a bottom surface of the cell chip accommodation unit 100 maybe identical to or higher than a level h2 of the drug stored in the drug storage unit 200. In this case, a level h1 of the drug filled in the cell chip accommodation unit 100 may be higher than the level h2 of the drug storage unit 200 all the time, and thus the transfer of drug may be easily undertaken from the cell chip accommodation unit 100 to the drug storage unit 200.

Another embodiment of the present invention will now be described with reference to FIGS. 11 and 12.

According to another embodiment of the present invention, as shown in FIG. 11, the cell chip accommodation unit 100 may be divided into a plurality of regions. In this regard, respective regions may accommodate different cell chips therein. In addition, each region may be independently connected to the drug storage unit 200. To this end, the number of regions of the cell chip accommodation unit 100 may be the same as that of the drug storage spaces 210, 212, 214, and 216 of the drug storage unit 200. That is, the cell chip accommodation unit 100 may be divided into four regions being the same as the drug storage spaces 210, 212, 214, and 216 of FIG. 11.

According to another embodiment of the present invention, the drug storage unit 200 may have a cover. In more detail, the drug storage unit 200 may have a hole 204 formed in an upper portion thereof or an upper surface of the drug storage unit 200 is completely open as shown in FIG. 12. The drug storage unit 200 may further include a cover (not shown) covering the hole 204 or the open upper surface. In this regard, the cover may have any forms as long as the hole 204 or the open upper surface may be closed thereby. For example, the cover may be in a film form in such a manner that the cover may be adhered to or attached to the hole 204.

The drug storage unit 200 configured as above may selectively open or close the upper portion, thereby facilitating introduction or supplementation of a drug.

As set forth above, according to embodiments of the invention, a small quantity of drug can be uniformly and accurately circulated. Thus, the cell culture device according to embodiments of the present invention can quantitatively control a drug.

While the present invention has been shown and described in connection with the embodiments, it will be apparent to those skilled in the art that modifications and variations can be made without departing from the spirit and scope of the invention as defined by the appended claims.

What is claimed is:

1. A cell culture device comprising:
a cell chip accommodation unit accommodating a cell chip therein;
a drug storage unit storing a drug and having a closed structure;
a first pipe connecting the drug storage unit and the cell chip accommodation unit and transferring the drug from the drug storage unit to the cell chip accommodation unit;
a second pipe connecting the drug storage unit and the cell chip accommodation unit and transferring the drug from the cell chip accommodation unit to the drug storage unit; and
a pump circulating the drug between the cell chip accommodation unit and the drug storage unit.

2. The cell culture device of claim 1, wherein the first pipe is connected to a lower portion of the drug storage unit, and the second pipe is connected to an upper portion of the drug storage unit.

3. The cell culture device of claim 1, wherein the first pipe is connected to one side of the cell chip accommodation unit, and the second pipe is connected to the other side of the cell chip accommodation unit.

4. The cell culture device of claim 3, wherein the first pipe is connected to a first connection tube formed in a bottom of the cell chip accommodation unit, and the second pipe is connected to a second connection tube formed in the bottom of the cell chip accommodation unit.

5. The cell culture device of claim 4, wherein the first connection tube and the second connection tube have an average diameter greater than an average diameter of the first pipe and the second pipe.

6. The cell culture device of claim 1, wherein the cell chip accommodation unit includes a flow path determining a transfer path of the drug.

7. The cell culture device of claim 6, wherein the flow path has a zigzag shape.

8. The cell culture device of claim 1, wherein the drug storage unit is divided into a plurality of drug storage spaces such that the same or different kinds of drug are stored separately.

9. The cell culture device of claim 8, wherein the cell chip accommodation unit is divided into a plurality of regions.

10. The cell culture device of claim 9, wherein the plurality of regions and the plurality of drug storage spaces are equal in number.

11. The cell culture device of claim 1, wherein at least one of the cell chip accommodation unit and the drug storage unit includes a filter filtering foreign materials contained in the drug circulating between the cell chip accommodation unit and the drug storage unit.

12. The cell culture device of claim 1, wherein the drug storage unit includes a cover or a film member covering one surface thereof.

13. A cell culture device comprising:
a cell chip accommodation unit accommodating a cell chip therein;
a drug storage unit storing a drug and having a hydraulic head lower than that of the cell chip accommodation unit;
a first pipe connecting the drug storage unit and the cell chip accommodation unit and transferring the drug from the drug storage unit to the cell chip accommodation unit;
a second pipe connecting the drug storage unit and the cell chip accommodation unit and transferring the drug from the cell chip accommodation unit to the drug storage unit; and
a pump circulating the drug between the cell chip accommodation unit and the drug storage unit.

14. The cell culture device of claim 13, wherein the first pipe is connected to a lower portion of the drug storage unit, and the second pipe is connected to an upper portion of the drug storage unit.

15. The cell culture device of claim 13, wherein the first pipe is connected to one side of the cell chip accommodation unit, and the second pipe is connected to the other side of the cell chip accommodation unit.

16. The cell culture device of claim 15, wherein the first pipe is connected to a first connection tube formed in a bottom of the cell chip accommodation unit, and the second pipe is connected to a second connection tube formed in the bottom of the cell chip accommodation unit.

17. The cell culture device of claim 16, wherein the first connection tube and the second connection tube have an average diameter greater than an average diameter of the first pipe and the second pipe.

18. The cell culture device of claim 13, wherein the cell chip accommodation unit includes a flow path determining a transfer path of the drug.

19. The cell culture device of claim 18, wherein the flow path has a zigzag shape.

20. The cell culture device of claim 13, wherein the drug storage unit is divided into a plurality of drug storage spaces such that the same or different kinds of drug are stored separately.

21. The cell culture device of claim 20, wherein the cell chip accommodation unit is divided into a plurality of regions.

22. The cell culture device of claim 21, wherein the plurality of regions and the plurality of drug storage spaces are equal in number.

23. The cell culture device of claim 13, wherein at least one of the cell chip accommodation unit and the drug storage unit includes a filter filtering foreign materials contained in the drug circulating between the cell chip accommodation unit and the drug storage unit.

24. The cell culture device of claim 13, wherein the drug storage unit includes a cover or a film member covering one surface thereof.
